(19) United States
(12) Patent Application Publication
(10) Pub. No.: US 2021/0290877 A1
(43) Pub. Date: Sep. 23, 2021

(54) VENTILATOR INFORMATION DISPLAY METHOD AND APPARATUS, VENTILATOR, AND COMPUTER STORAGE MEDIUM

(71) Applicant: SHENZHEN MINDRAY BIO-MEDICAL ELECTRONICS CO., LTD., Shenzhen (CN)

(72) Inventors: Jiping HUANG, Shenzhen (CN); Ruiling PAN, Shenzhen (CN); Chenghua HUANG, Shenzhen (CN)

(73) Assignee: SHENZHEN MINDRAY BIO-MEDICAL ELECTRONICS CO., LTD., Shenzhen (CN)

(21) Appl. No.: 17/339,958

(22) Filed: Jun. 5, 2021

Related U.S. Application Data
(63) Continuation of application No. PCT/CN2018/119607, filed on Dec. 6, 2018.

(51) Int. Cl. A61M 16/00

(52) U.S. Cl. CPC A61M 16/024; A61M 16/0051; A61M 2205/52; A61M 2205/502; A61M 2205/35

(57) ABSTRACT

A ventilator information display method and apparatus, a ventilator, and a computer storage medium are provided. The method includes: receiving a patient type; and outputting, according to the received patient type and pre-stored correspondences between patient types and display information, corresponding display information. That is, according to the received patient type, corresponding display information can be obtained; namely, for patients of different types, different ventilator information can be displayed. Because each patient type has its own unique physiological characteristics, the strategies for ventilator information for different patient types are also different. In this way, the method can meet actual needs and can improve the work efficiency of medical staff.

### CROSS-REFERENCE OF RELATED APPLICATIONS

This is a bypass continuation application of International Application No. PCT/CN2018/119607, entitled “VENTILATOR INFORMATION DISPLAY METHOD AND APPARATUS, VENTILATOR, AND COMPUTER STORAGE MEDIUM,” filed Dec. 6, 2018, the content of which is incorporated by reference herein in its entirety.

### TECHNICAL FIELD

The disclosure relates to patient information presentation technology, and in particular to a ventilator information display method and apparatus, a ventilator, and a computer storage medium.

### BACKGROUND

Clinically, there are a variety of patient types, such as obese patients, the elderly and neonates; each patient type has its own unique physiological characteristics, and management strategies (e.g., perioperative anesthesia management strategies) for different patient types are also different. However, in the related art, when ventilator information (e.g., perioperative anesthesia management parameters) is displayed, it is not distinguished according to the pathological characteristics of patients, that is, the same ventilator information is displayed for different types of patients. For example, a ventilator provides the same UI interface the same display parameters and the same functions for obese patients, the elderly and neonates. In this way, the solution that the same ventilator information is displayed for different types of patients does not meet actual requirements, and reduces the working efficiency of medical staff.

### SUMMARY

In order to solve the above technical problems, an embodiment of the disclosure provides a ventilator information display method and apparatus, a ventilator, and a computer storage medium.

An embodiment of the disclosure provides a ventilator information display method, comprising:

receiving a patient type; and

outputting corresponding display information according to the received patient type and a pre-stored correspondence between patient types and display information.

An embodiment of the disclosure further provides a ventilator information display apparatus, comprising: a processor and a memory, wherein

the memory is configured to store a correspondence between patient types and display information; and

the processor is configured to receive a patient type, and to output corresponding display information according to the received patient type and the correspondence between patient types and display information read from the memory.

An embodiment of the disclosure further provides another ventilator information display apparatus, comprising: a receiving unit and a processing unit, wherein

the receiving unit is configured to receive a patient type; and

the processing unit is configured to output corresponding display information according to the patient type received by the receiving unit and a pre-stored correspondence between patient types and display information.

An embodiment of the disclosure further provides a ventilator comprising any one ventilator information display apparatus described above.

An embodiment of the disclosure further provides a computer storage medium with a computer program stored thereon, wherein any one ventilator information display apparatus described above is implemented when the computer program is executed by a processor.

In the technical solutions provided by the embodiments of the disclosure, the patient type is received; and corresponding display information is output according to the received patient type and a pre-stored correspondence between patient types and display information. In other words, the corresponding display information may be obtained according to the received patient type; that is, different ventilator information may be displayed for different types of patients. Since each patient type has its own unique physiological characteristics, the strategies for ventilator information for different patient types are also different. In this way, the technical solutions of the embodiments of the disclosure can meet practical requirements, and the working efficiency of medical staff can be improved.

### BRIEF DESCRIPTION OF THE DRAWINGS

FIG. 1 is a flow chart of a ventilator information display method according to an embodiment of the disclosure;

FIG. 2 is a schematic diagram of a patient type setting interface according to an embodiment of the disclosure;

FIG. 3 is a partial enlarged schematic diagram of FIG. 2;

FIG. 4 is a schematic diagram of a UI interface customized for obese patients according to an embodiment of the disclosure;

FIG. 5 is a schematic diagram of a UI interface customized for old patients according to an embodiment of the disclosure;

FIG. 6 is a schematic diagram of a UI interface customized for neonate patients according to an embodiment of the disclosure;

FIG. 7 is a schematic graph of a change curve of an airway pressure of a patient in an embodiment of the disclosure;

FIG. 8 is a schematic graph of a change curve of a tidal volume in an embodiment of the disclosure;

FIG. 9 is a structural schematic diagram of the constitution of a ventilator information display apparatus according to an embodiment of the disclosure; and

FIG. 10 is a structural schematic diagram of another ventilator information display apparatus according to an embodiment of the disclosure.

### DESCRIPTION OF THE PREFERRED EMBODIMENTS

In order to understand the features and technical contents of embodiments of the disclosure in more detail, the implementation of the embodiments of the application will be described below in detail with reference to the accompanying drawings, which are for reference and illustration only, and are not intended to limit the embodiments of the disclosure.

When ventilator information is displayed, each patient type has its own unique physiological and pathological characteristics, and the ventilator information needing to be displayed is different for different patient types. For example, in the field of perioperative anesthesia management, each patient type has its own unique physiological and pathological characteristics, and perioperative anesthesia management strategies are different for different patient types. The following is an illustrative description of several different patient types.

Obese patients have their unique pathophysiological characteristics in which the characteristics of a respiratory system include the following aspects:

1. Decreased Functional Residual Capacity: obesity may affect diaphragmatic and thoracoabdominal movements, resulting in decreased functional residual capacity, regional atelectasis and increased intrapulmonary shunt; these changes are more significant by general anesthesia, and after anesthesia, the functional residual capacity in obese patients decreases by 50%, but that in non-obese patients decreases by only 20%. The decreased functional residual capacity impairs the ability of obese patients to tolerate apnea.

2. Decreased Lung Compliance: the accumulation of fat on the chest wall and abdomen and an increase in pulmonary artery blood volume leads to a decrease in pulmonary compliance and an increase in airway resistance; when obese patients are in a supine position, the decrease in pulmonary compliance and the increase in airway resistance are more significant; and a few patients with morbid obesity and cardiac dysfunction cannot tolerate the supine position at all. The supine position may lead to fatal cardiopulmonary failure, which is called obesity supine death syndrome.

3. Increased Resting Metabolic Rate, Oxygen Consumption and Work of Breathing: oxygen consumption and carbon dioxide production increase as weight gains, obese patients need to increase minute ventilation to maintain normal carbon dioxide level in the blood, resulting in increased work of breathing.

4. Obstructive Sleep Apnea (OSA): OSA is defined such that the duration of apnea during sleep is greater than 10 seconds, and there may be frequent apneas and hypopneas during sleep; and obesity is the most important risk factor leading to sleep apnea. Obese patients periodically experience partial or complete upper airway obstruction during sleep, and may suffer from frequent apneas and hypopneas.

In view of the unique characteristics of the respiratory system of the obese patients, the anesthesia management of obese patients needs to provide targeted perioperative anesthesia management strategies according to the pathophysiological characteristics of the respiratory system. For obese patients under general anesthesia, due to their characteristics such as high oxygen consumption, low oxygen storage capacity, faster decrease in oxygen saturation after breathing stops and difficult airways, intraoperative anesthesia management has the following requirements:

1. Anesthesia induction period: during endotracheal intubation, a technique of nasal administration of high flow oxygen (15 L/min to 70 L/min) is needed to prolong the hypoxia time of the patients.

2. Anesthesia maintenance: it is preferred to use a drug with minimal accumulation in the adipose tissue; and continuous propofol infusion or an inhalational anesthetic can be used for anesthesia maintenance, and desflurane and sevoflurane with a low blood gas distribution coefficient are better than isoflurane.

3. Ventilation management: the two most important issues are pulmonary oxygenation function and airway pressure; with regard to mechanical ventilation, a volume control or pressure control mode is applicable, and appropriate increase in inspired oxygen concentration (>50%) of patients and the use of low and middle levels of positive end expiratory pressure (PEEP) (5 cmH₂O to 10 cmH₂O) may be more helpful to improve the intraoperative and postoperative oxygenation functions of obese patients; and the combination of intermittent lung inflation and the PEEP may be effective for patients who are still difficult to maintain adequate oxygenation with high-oxygen-concentration ventilation during operation.

4. Anesthesia monitoring: obese patients need to undergo routine electrocardiogram, peripheral oxygen saturation, non-invasive blood pressure, and end-tidal carbon dioxide monitoring; and the depth of anesthesia is monitored using a bispectral index (BIS), especially under total intravenous anesthesia, in order to avoid overdose of anesthetic drugs. It is recommended to use muscle relaxation monitoring during operation.

The elderly also has typical physiological and pathological characteristics, which are exemplified in several respects as follows:

1. The lung function of old patients declines with aging, and the lung function of those with chronic respiratory diseases or recent acute respiratory diseases will be further impaired; and for an early warning index of the fragile lung function: airway pressure, in a state of relatively constant tidal volume, under anesthesia, surgery and/or drugs, the patient's airway may be more susceptible to changes in lung volume (such as body position changes, pneumoperitoneum, thoracic collapse, and one-lung ventilation), airway spasm, or increased pressure caused by factors such as increased lung water, which should be analyzed and treated according to the causes of diseases.

2. The old patients need comprehensive approaches to implement intraoperative lung function protection, and the measures include:

(1) mechanically ventilated patients implement a low tidal volume+moderate PEEP (5-8 cmH₂O) strategy, and the low tidal volume is standard body weight×6-8 ml/kg; and 3-5 consecutive manually controlled lung distentions are given per hour, and a pressure of lung distention is not more than 30 cmH₂O, which also help to prevent postoperative atelectasis; and

(2) FiO₂ should not exceed 60% in order to prevent absorptive atelectasis.

3. Due to the decline of brain function in old patients, especially the increase in old patients with fragile brain function, the decline of liver and kidney functions leads to decreased drug metabolism. These comprehensive factors remarkably increase the sensitivity of old patients to sedative and analgesic drugs during operation. Therefore, it is very important to strengthen the monitoring of the sedation depth of anesthesia, to avoid intraoperative awareness caused by excessive sedation and insufficient anesthesia of the patients.

According to the physiological and pathological characteristics of the elderly, intraoperative anesthesia management strategies may be provided as follows:

1. Early warning is provided: in a volume ventilation mode, when a set tidal volume value does not change, and an airway pressure monitored during operation significantly rises, an alarm prompt may be given.

2. A pulmonary reexpansion function, which can be started regularly, is provided specially for the elderly.

3. Anesthesia depth monitoring (BIS monitoring) is routinely provided during operation.

The neonate patient type also has typical physiological and pathological characteristics, which are exemplified in several respects as follows:

1. The incidence of retinopathy is high in neonates, especially in premature infants with low birth weight. Oxygen-air mixed inhalation may be used to avoid lesions in a perioperative period, and pure oxygen inhalation is not recommended. Long-term high concentrations (FiO₂>40%) is prone to cause retinopathy.

2. N₂O is not suitable for anesthesia in neonates with low body weight and endoscopic surgery.

3. It is advisable to choose a cuff-free tracheal catheter, a pressure-limited timing ventilation mode is generally selected, a ventilation pressure, a respiratory rate and an oxygen concentration are adjusted, and an end-expiratory CO₂ partial pressure is closely monitored to maintain it in a range of 35-40 mmHg.

4. Their metabolism is vigorous, the oxygen demand is large, and their needs are mainly met by increasing the respiratory rate during oxygen deficiency.

5. If sick infants do not have an open venous access before induction, induced inhalation anesthesia is usually used, and sevoflurane, which does not stimulate the airway and is stable and rapid in induction, is currently an inhaled induction drug commonly used in clinical practice.

6. Once circulation or severe suppression of respiration occurs during induction of anesthesia, the concentration of the inhaled anesthetic should be reduced immediately, or the inhaled anesthetic should be completely stopped, and flushing should be performed using 100% high-flow oxygen.

According to the physiological and pathological characteristics of neonates, intraoperative anesthesia management strategies may be provided as follows:

1. The oxygen concentration does not exceed 40%.

2. For cuff-free intubation, a pressure ventilation mode is selected by default.

3. A high respiratory rate is default.

4. Sevoflurane is commonly used as the inhaled anesthetic.

5. A high flow rate oxygen therapy function, etc. is provided.

In the related art, ventilators such as anesthesia machines cannot be distinguished according to the pathophysiological characteristics of different patient types, that is, the same ventilator information is provided for different patient types, so that information display needs of patients of a specific type are not met, for example, for obese patients, a doctor needs to manually adjust ventilation parameters suitable for obese patients; the anesthesia machines cannot provide a high-flow-rate oxygen therapy function, the doctor needs to find other high-flow-rate oxygen therapy equipment, or choose other solutions to deal with difficult tracheal intubation of the obese patients; and anesthesia machine functions or tools commonly used during anesthesia of the obese patients are scattered on the anesthesia machines, and even some commonly used functions are hidden in menus and are not easy to enable, affecting the working efficiency of the doctor.

Aiming to solve the above technical problems, an embodiment of the disclosure provides a ventilator information display method, which can be applied to a ventilator. Herein, the ventilator may be an anesthesia machine or other ventilators.

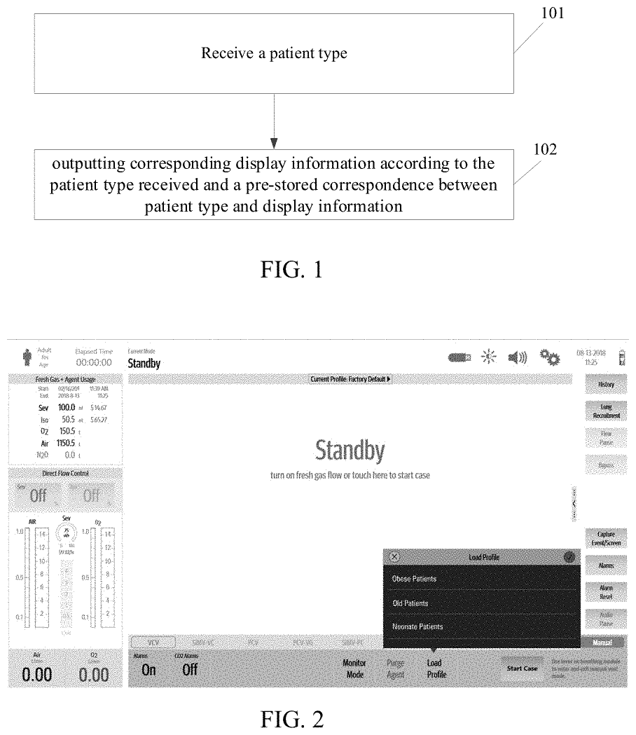

FIG. 1 is a flow chart of a ventilator information display method according to an embodiment of the disclosure. As shown in FIG. 1, the flow may comprise the following steps.

At step 101, a patient type is received.

In practical applications, a ventilator, such as an anesthesia machine, may provide a human-machine interaction interface, and various patient types may be displayed on the human-machine interaction interface. Alternatively, various patient types may be displayed through a view; the patient type for which ventilator information needs to be displayed may then be determined by receiving the patient type selected by a user (e.g., by the user clicking on the corresponding patient type); and the patient type for which ventilator information needs to be displayed may be known by medical staff in advance.

Herein, various types of patients may be classified according to at least one of physiological characteristics and surgery types, for example, obese patients, old patients, neonate patients and so on may be classified according to physiological characteristics; and depending on surgery types, laparoscopic surgery patients, cardiac surgery patients and so on may be classified.

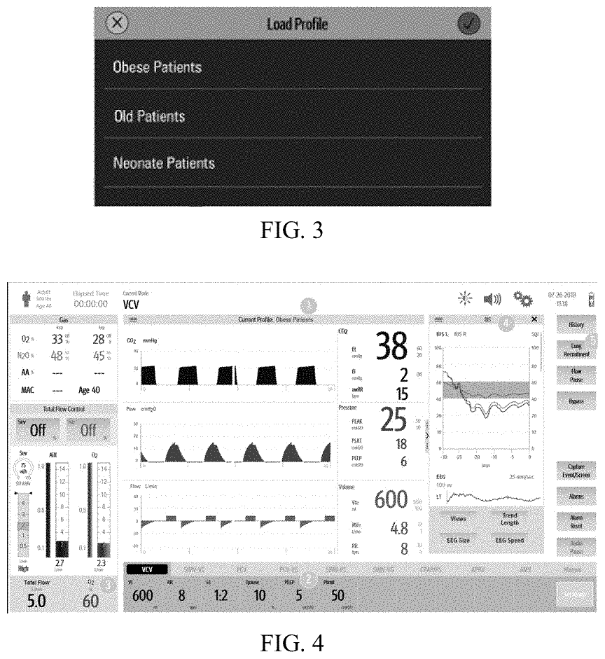

FIG. 2 is a schematic diagram of a patient type setting interface according to an embodiment of the disclosure, and FIG. 3 is a partial enlarged schematic diagram of FIG. 2. Referring to FIGS. 2 and 3, the patient type setting interface is a human-machine interaction interface. In actual implementation, the medical staff can set patient types by loading a load profile, and as an example, three patient types are provided in FIGS. 2 and 3 for selection. The three patient types are Obese patients, Old patients and Neonate patients, respectively. It should be noted that FIGS. 2 and 3 are only illustrative description of the patient types, and that the patient types in the embodiments of the disclosure are not limited to the three patient types described above, but may be other patient population types having the same physiological and pathological characteristics.

At step 102, Corresponding display information is output according to the received patient type and a pre-stored correspondence between patient types and display information.

In practical applications, display information corresponding to various types of patients may be predetermined according to pathological characteristics of the various types of patients. For example, the display information corresponding to the obese patients, the old patients and the neonate patients may be respectively determined according to the pathological characteristics of the obese patients, the old patients and the neonate patients. The display information corresponding to each patient type herein includes, but is not limited to, perioperative anesthesia management parameters and other information.

Further, the display information corresponding to the various types of patients may also be stored after being determined, for example, the display information corresponding to the various types of patients may be stored in a storage medium of the ventilator. Specifically, after the correspondence between patient types and display information is stored for the first time, if a correspondence between patient types and display information is received again, the stored correspondence between patient types and display information may be updated or modified according to the currently received correspondence between patient types and display information.

Optionally, the display information may include one or more of patient information, monitoring information, control information, and layout information.

Herein, the patient information may include name, age, sex and other information of a patient, and the display of the patient information may be realized in the form of numerical values, characters and so on in actual implementation; the monitoring information may include one or more of real-time values, short trends, long trends and mean values of physiological parameters of a patient, and monitoring display may be presented through a numerical value change curve in actual implementation; the control information may include one or more of ventilation control information, ventilator control information and a commonly used tool, and the control information may be presented through a shortcut key and the like in actual implementation; and the layout information is used to indicate a distribution mode of the display information on a display interface. The physiological parameters of a patient include exhaled CO2, anesthesia depth, airway pressure, etc.

As an example, the ventilation control information may include a ventilation mode, ventilation parameters and so on of a ventilator, each ventilation parameter may also have a preset default value (e.g., a default oxygen concentration value); the ventilator control information includes information for controlling the ventilator, such as ventilator system setting, display setting, alarm setting and so on; the commonly used tool includes a shortcut key and so on. In an example, the shortcut key may indicate whether a pulmonary reexpansion function of the ventilator is enabled, that is, the pulmonary reexpansion function may be enabled or disabled by clicking the shortcut key. In another example, the shortcut key may represent an alarm setting button, and it may be determined whether an early warning function, a ventilator display setting and so on are enabled by clicking the shortcut key. The shortcut key may be configured and modified according to user needs.

In the embodiment of the disclosure, the display information may be output on a user interface (UI). In a specific example, the ventilator may present a UI interface after receiving a patient type selected by the user, and display output display information using the UI interface. Herein, each patient type has a corresponding customized UI interface and its functional arrangement mode according to the layout information. As can be seen, when receiving the patient type selected by the user, the ventilator may switch from one interface to a UI interface (which is a main function and monitoring interface corresponding to the current patient type) customized for the current patient type. Optionally, the current patient type may be displayed in the UI interface customized for each patient type, for example, the current patient type may be displayed over a waveform display area of the UI interface.

In a specific example of the above display information, functions (tools) commonly used by a current patient are displayed in the form of buttons in a main interface shortcut key area of the UI interface (e.g., pulmonary reexpansion function buttons for obese patients and old patients, which are convenient for a doctor to use), and a monitoring parameter interface necessary for the current patient is displayed on the UI interface and is in an On state by default (e.g., a BIS monitoring interface).

Further, it is also possible to determine whether the output display information meets a preset alarm condition, and if the output display information meets the preset alarm condition, early warning prompt information may be generated.

The early warning condition herein may be an alarm condition set for one or more of a patient type, a human organ, a surgery type, etc., and may, for example, an early warning function provided for a fragile lung function. In a specific example, an early warning function for excessive pressure changes or an early warning function for excessive tidal volume changes may be provided for the fragile lung function. The early warning function for excessive pressure changes is provided for old patients, and in a volume ventilation mode, when a set tidal volume value does not change, while an airway pressure monitored during operation significantly rises, an early warning prompt is given. The early warning function for excessive tidal volume changes is provided for neonate patients, and in a pressure ventilation mode, when a set pressure value does not change, while a tidal volume monitored during operation significantly decreases, an early warning prompt is given. The mode of the early warning prompt may be an alarm mode or a pressure and tidal volume short trend display mode.

Display modes of ventilator information are illustrated below for several different patient types.

Based on step 101, when the received patient type selected by the user is obese patients, the UI interface customized for obese patients may be presented.

FIG. 4 is a schematic diagram of a UI interface customized for obese patients according to an embodiment of the disclosure, and portions marked respectively with \textcircled{1}, \textcircled{2}, \textcircled{3}, \textcircled{4} and \textcircled{5} in FIG. 4 will be described below.

\textcircled{1} The current patient type is displayed as obese patients.

\textcircled{2} A set of special default values of ventilation parameters are provided for obese patients, for example, PEEP=5 cmH₂O, low and middle levels of PEEP may be more conducive to intraoperative and postoperative oxygenation functions of the obese patients; \textcircled{3} a default inspired oxygen concentration for the obese patients is 60% or more (the obese patients have high oxygen consumption, and the inspired oxygen concentration needs to be above 50%).

\textcircled{4} BIS anesthesia depth monitoring is displayed by default on a main interface (which is a main interface of the UI interface customized for obese patients), and the BIS monitoring of anesthesia depth is recommended for obese patients, especially those under total intravenous anesthesia, to avoid overdose of anesthetic drugs.

\textcircled{5} The pulmonary reexpansion function is displayed by default in a shortcut key area of the main interface (which is the main interface of the UI interface customized for obese patients), and in the case where the obese patient is difficult to maintain sufficient oxygenation, a ventilation mode of combining intermittent expansion and PEEP is needed.

Based on step 101, when the received patient type selected by the user is old patients, the UI interface customized for old patients may be presented.

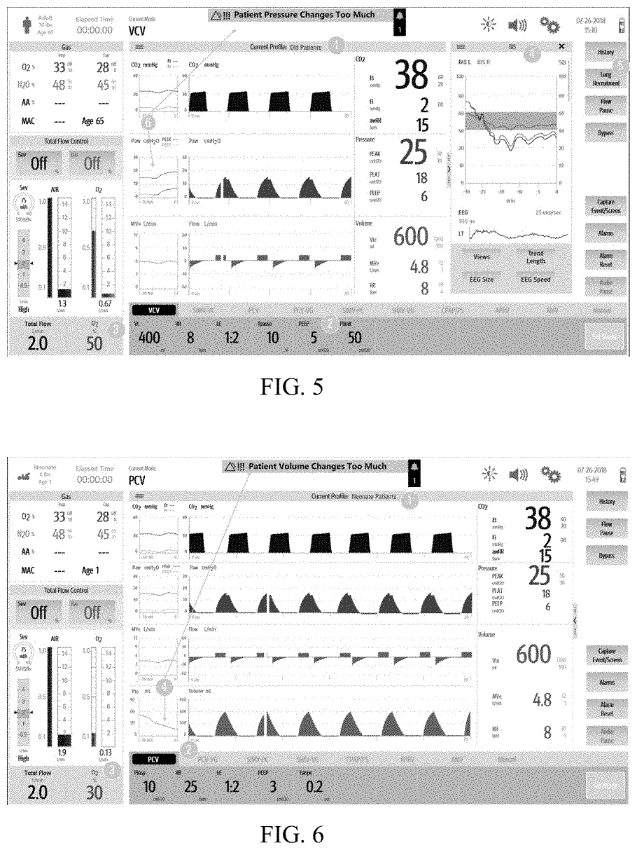

FIG. 5 is a schematic diagram of a UI interface customized for old patients according to an embodiment of the disclosure, and portions marked respectively with \textcircled{1}, \textcircled{2}, \textcircled{3}, \textcircled{4}, \textcircled{5} and \textcircled{6} in FIG. 5 are described below.

\textcircled{1} The current patient type is displayed as old patients.

\textcircled{2} A set of special default values of ventilation parameters are provided for old patients, for example, a default value of Vt is standard body weight×6 ml/kg, PEEP=5 cmH₂O, an inspiration/expiration ratio is 1:2.

\textcircled{3} A default inspired oxygen concentration for the old patients is 50% (the inspired oxygen concentration of the old patients cannot exceed 60% in order to prevent absorptive atelectasis).

\textcircled{4} BIS anesthesia depth monitoring is displayed by default on a main interface (which is a main interface of the UI interface customized for old patients), a drug metabolism function of the old patients is reduced due to liver function degradation, the sensitivity of the old patients to sedation and analgesia drugs during operation is remarkably increased, and anesthesia sedation depth monitoring needs to be strengthened.

\textcircled{5} A pulmonary reexpansion function is displayed by default in a shortcut key area of a main interface (which is a main interface of the UI interface customized for old patients), and for the old patients, 3-5 lung distentions need to be given per hour, and the lung distention pressure does not exceed 30 cmH2O, facilitating prevention of absorption atelectasis.

\textcircled{6} The early warning function for a fragile lung function is provided, and in a volume ventilation mode, when the set tidal volume value does not change, while the airway pressure monitored during operation significantly rises (changes in the pressure in a period of time may be displayed through a short trend function for the pressure), an alarm prompt may be given. The lung function of old patients declines with aging, and the patient's airway may be more susceptible to changes in lung volume (such as body position changes, pneumoperitoneum, thoracic collapse, and one-lung ventilation), airway spasm, or increased pressure caused by factors such as increased lung water, which should be analyzed and treated according to the causes of diseases.

Based on step 101, when the received patient type selected by the user is neonate patients, a UI interface customized for neonate patients may be presented.

FIG. 6 is a schematic diagram of a UI interface customized for neonate patients according to an embodiment of the disclosure, and portions marked respectively with \textcircled{1}, \textcircled{2}, \textcircled{3} and \textcircled{4} in FIG. 6 are described below.

\textcircled{1} The current patient type is displayed as neonate patients.

\textcircled{2} A default ventilation mode for the type of neonate patients is a PCV mode (cuff-free tracheal catheters are generally selected for neonates, so the pressure ventilation mode is selected), and a volume ventilation mode VCV and an SIMV-VC mode are no longer displayed in a ventilation mode area. In addition, a set of special default values of ventilation parameters are provided for neonates, such as a default high respiration rate of 25 bpm.

\textcircled{3} A default inspired oxygen concentration of neonate patients is 30%, and the incidence of neonatal retinopathy is high, so that the oxygen concentration is not recommended to exceed 40% for a long time. If the set oxygen concentration of the anesthesia machine exceeds 40%, prompting information needs to be given, and the user can adjust the set value to be higher after confirmation.

\textcircled{4} The early warning function for excessive tidal volume changes is provided, and in the pressure ventilation mode, when a set pressure value does not change, while a tidal volume monitored during operation significantly decreases (changes in the tidal volume in a period of time may be displayed through a short trend function for the tidal volume), an alarm prompt may be given. Neonate body position changes and other cases may occur in the same pressure ventilation mode, the actual tidal volume entering the lung of a patient changes greatly, the tidal volume at the moment cannot meet the ventilation need of the neonate, and therefore an alarm may be given to remind a doctor to perform corresponding treatment on the patient.

In actual implementation, a change curve of perioperative anesthesia management parameters corresponding to the alarm condition may be displayed on the interface, and the early warning function for a fragile lung will be exemplified below.

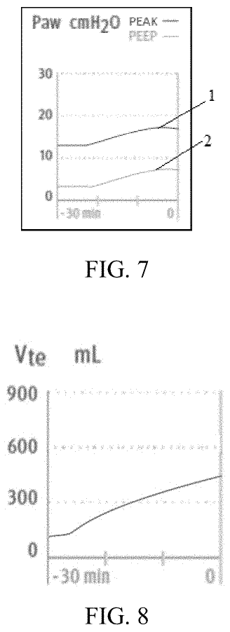

FIG. 7 is a schematic graph of a change curve of an airway pressure of a patient in an embodiment of the disclosure. As shown in FIG. 7, in the volume ventilation mode, when the set tidal volume value does not change, while the airway pressure monitored during operation changes significantly (increases or decreases), an early warning prompt may be given. The mode of the early warning prompt may be an alarm mode or a short trend mode shown in FIG. 7. FIG. 7 provides a short trend chart of a peak pressure PEAK and a positive end-expiratory pressure PEEP, wherein the curve 1 represents a short trend of the peak pressure PEAK, and the curve 2 represents a short trend of the positive end-expiratory pressure PEEP. Referring to FIG. 7, a short trend of a pressure parameter may be monitored over a period of time (e.g., 30 min, 1 h, 2 h, etc.), and a threshold value for excessive change warning may be a fixed value or a user-settable value.

FIG. 8 is a schematic graph of a change curve of a tidal volume in an embodiment of the disclosure. As shown in FIG. 8, in the pressure ventilation mode, when the set pressure value does not change, while the tidal volume monitored during operation changes significantly (increases or decreases), an early warning prompt may be given. The mode of the early warning prompt may be an alarm mode or a short trend mode shown in FIG. 8. A short trend chart of an expiratory tidal volume Vte is provided, which can monitor a short trend of a tidal volume parameter over a period of time (e.g., 30 min, 1 h, 2 h, etc.), and a threshold value for excessive change warning may be a fixed value or a user-settable value.

In practical applications, step 101 may be implemented by a processor of the ventilator, which may be at least one of an application specific integrated circuit (ASIC), a digital signal processor (DSP), a digital signal processing device (DSPD), a programmable logic device (PLD), a field programmable gate array (FPGA), a central processing unit (CPU), a controller, a microcontroller and a microprocessor. Step 102 may be implemented by the processor in the ventilator in combination with a display.

According to the embodiment of the disclosure, the patient types may be subdivided, specifically, the patient types are distinguished according to the physiological and pathological characteristics of patients, a set of effective ventilator information is provided for each patient type with the same physiological and pathological characteristics and may be presented through a corresponding customized UI, and thus the ventilator information may be adaptively displayed according to the patient types.

On the basis of the ventilator information display method provided by the embodiment of the disclosure, an embodiment of the disclosure further provides a ventilator information display apparatus, which may be applied to a ventilator.

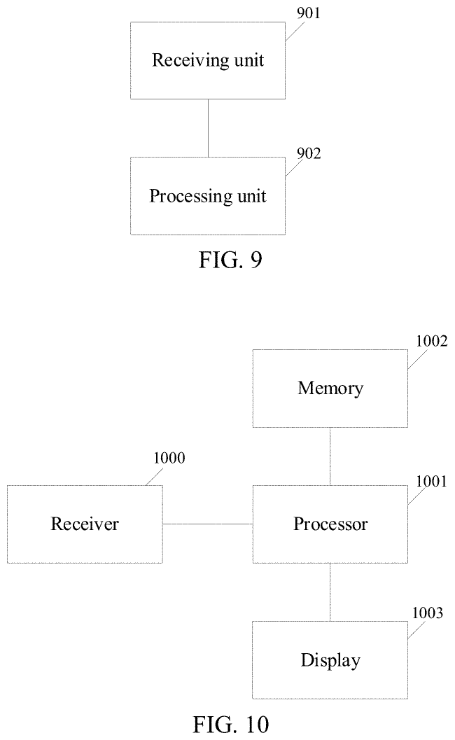

FIG. 9 is a structural schematic diagram showing the constitution of a ventilator information display apparatus according to an embodiment of the disclosure. As shown in FIG. 9, the apparatus comprises a receiving unit 901 and a processing unit 902, wherein

the receiving unit 901 is configured to receive a patient type;

and the processing unit 902 is configured to output corresponding display information according to the patient type received by the receiving unit and a pre-stored correspondence between patient types and display information.

In an embodiment, the display information includes one or more of patient information, monitoring information, control information, and layout information.

In an embodiment, the monitoring information includes one or more of real-time values, short trends, long trends and mean values of physiological parameters of a patient.

In an embodiment, the control information includes one or more of ventilation control information, ventilator control information, and a commonly used tool.

In an embodiment, the patient type is associated with at least one of physiological characteristics and surgery types.

In an embodiment, the processing unit 902 is further configured to generate early warning prompt information when the output display information satisfies a preset alarm condition.

In practical applications, the receiving unit 901 may be implemented by a CPU, a micro processor unit (MPU), a DSP, or an FPGA located in the ventilator, and the processing unit 902 may be implemented by the CPU, the MPU, the DSP, or the FPGA located in the ventilator in combination with the display.

In addition, various functional module in this embodiment may be integrated into one processing unit, various units may be physically present separately, or two or more units may be integrated into one unit. The integrated units described above may be implemented in the form of hardware or software functional modules.

If the integrated units are implemented in the form of the software functional modules but not sold or used as independent products, they may be stored in a computer-readable storage medium. Based on such an understanding, the technical solution of this embodiment, in essence, or its part contributing to the prior art, or all or part of the technical solution may be embodied in the form of a software product. The computer software product is stored in a storage medium and includes a number of instructions for causing a computer device (which may be a personal computer, a server, or a network device) or a processor to execute all or some of the steps of the method according to this embodiment. The foregoing storage medium includes: a U disk, a removable hard disk, a read only memory (ROM), a random access memory (RAM), a magnetic disk or an optical disk and other media that can store program codes.

In particular, computer program instructions corresponding to a ventilator information display method in this embodiment may be stored in a storage medium such as an optical disk, a hard disk and a U disk, and when the computer program instructions corresponding to a ventilator information display method in the storage medium are read or executed by an electronic device, any one of the ventilator information display methods of the previous embodiments is implemented.

Based on the same technical concept of the previous embodiments, referring to FIG. 10, another ventilator information display apparatus provided by an embodiment of the disclosure is shown. The apparatus may comprise: a processor 1001 and a memory 1002, wherein

the memory 1002 is configured to store a correspondence between patient types and display information; and

the processor 1001 is configured to receive a patient type, and to output corresponding display information according to the received patient type and the correspondence between patient types and display information read from the memory.

In an embodiment, the display information includes one or more of patient information, monitoring information, control information, and layout information.

In an embodiment, the monitoring information includes one or more of real-time values, short trends, long trends and mean values of physiological parameters of a patient.

In an embodiment, the control information includes one or more of ventilation control information, ventilator control information, and a commonly used tool.

In an embodiment, the apparatus further comprises:

a receiver 1000 configured to receive an input correspondence between patient types and display information, wherein

the processor 1001 is further configured to update the correspondence between patient types and display information stored in the memory according to the received correspondence between patient types and display information.

Specifically, after the correspondence between patient types and display information is stored for the first time, the processor may be configured to, when receiving a correspondence between patient types and display information again, update or modify the stored correspondence between patient types and display information according to the currently received correspondence between patient types and display information.

In an embodiment, the patient type is associated with at least one of physiological characteristics and surgery types.

In an embodiment, the processor 1001 is further configured to generate early warning prompt information when the output display information satisfies a preset alarm condition.

In an embodiment, the apparatus further comprises a display 1003, wherein the display 1003 is configured to display, under control of the processor, the display information through a user interface.

In practical applications, the memory 1002 may be a volatile memory such as an RAM, a non-volatile memory such as an ROM, a flash memory, a hard disk drive (HDD) or a solid-state drive (SSD), or a combination there, and provide instructions and data to the processor 1001.

The processor 1001 may be at least one of an ASIC, a DSP, a DSPD, a PLD, an FPGA, a CPU, a controller, a microcontroller, and a microprocessor. It will be appreciated that for different devices, other electronics may also implement functions of the processor described above and are not specifically limited by the embodiments of the disclosure.

An embodiment of the disclosure also provides a ventilator comprising any one of ventilator information display apparatuses described above. Herein, the ventilator may be an anesthesia machine or other types of ventilators.

The technical solutions specified in the embodiments of the disclosure may be combined in any manner if no conflict is caused.

In the several embodiments provided by the disclosure, it should be understood that the disclosed methods and intelligent devices may be implemented in other ways. The embodiments of the ventilator described above are merely illustrative, for example, the division of the units is merely a logical functional division, and there may be other division modes in actual implementation, for example, multiple units or components may be combined, or may be integrated into another system, or some features may be omitted or not implemented. In addition, the mutual coupling or direct coupling or communication connection between the components as shown or discussed may be indirect coupling or communication connection through some interfaces, devices or units, and may be in electrical, mechanical or other forms.

The units described as separate parts may be or may not be physically separated, and parts displayed as units may be or may not be physical units, that is, they may be located in one position, or may be distributed on a plurality of network units. Some or all of the units may be selected according to actual needs to achieve the objectives of the solutions of the embodiments.

In addition, various functional units in various embodiments of the disclosure may be integrated into a second processing unit, or various units may respectively serve as a separate unit, or two or more units may be integrated into one unit. The foregoing integrated units may be implemented in the form of hardware or hardware and software functional units.

The above descriptions are merely specific embodiments of the disclosure, but the scope of protection of the disclosure is not limited thereto. Changes or substitutions readily figured out by those skilled in the art within the technical scope disclosed in the disclosure shall fall within the scope of protection of the application.

1-19. (canceled)

20. A ventilator information display method, comprising:
receiving a patient type; and
outputting corresponding display information according to the patient type received and a pre-stored correspondence between patient type and display information, wherein
the display information outputted includes control information, said control information comprises ventilation control information having a ventilation mode and ventilation parameters of the ventilator.

21. The ventilator information display method according to claim 20, wherein the display information outputted further includes one or more of: patient information, monitoring information, and layout information.

22. The ventilator information display method according to claim 21, wherein the monitoring information includes one or more of: real-time value, short trend, long trend and mean value of physiological parameters of a patient.

23. The ventilator information display method according to claim 20, wherein the control information further includes one or more of: ventilation control information, ventilator control information, and commonly used tool information.

24. The ventilator information display method according to claim 20, further comprising:
determining and storing display information corresponding to patients of each type according to pathological characteristics of said patients.

25. The ventilator information display method according to claim 24, further comprising:
classifying patients of each type in advance, according to at least one of physiological characteristic and surgery type.

26. The ventilator information display method according to claim 20, further comprising:
generating early warning prompt information when the display information outputted satisfies a preset alarm condition.

27. The ventilator information display method according to claim 20, wherein outputting the corresponding display information comprises:
displaying the display information through a user interface.

28. A ventilator information display apparatus, comprising: a processor and a memory, wherein
the memory is configured to store a correspondence between patient type and display information; and
the processor is configured to receive a patient type, and to output corresponding display information according to the patient type received and said correspondence between patient type and display information, wherein
the target display information includes control information, said control information comprises ventilation control information having a ventilation mode and ventilation parameters of the ventilator.

29. The ventilator information display apparatus according to claim 28, wherein the display information outputted further includes one or more of: patient information, monitoring information, and layout information.

30. The ventilator information display apparatus according to claim 29, wherein the monitoring information includes one or more of: real-time value, short trend, long trend and mean value of physiological parameters of a patient.

31. The ventilator information display apparatus according to claim 28, wherein the control information further includes one or more of: ventilation control information, equipment control information, and commonly used tool information.

32. The ventilator information display apparatus according to claim 28, further comprising:
a receiver, which is configured to receive an input correspondence between a patient type and a display information, wherein
the processor is further configured to update the correspondence between each patient type and corresponding display information stored in the memory according to the input correspondence between patient type and display information.

33. The ventilator information display apparatus according to claim 32, wherein the patient type is associated with at least one of physiological characteristic and surgery type.

34. The ventilator information display apparatus according to claim 28, wherein the processor is further configured to generate early warning prompt information when the display information outputted satisfies a preset alarm condition.

35. The ventilator information display apparatus according to claim 28, further comprising a display, wherein
the display is configured to display, under control of the processor, the display information through a user interface.

36. A ventilator information display apparatus, comprising: a receiving unit and a processing unit, wherein
the receiving unit is configured to receive a patient type; and
the processing unit is configured to output corresponding display information according to the patient type received by the receiving unit, and a pre-stored correspondence between patient type and display information, wherein
the display information outputted includes control information, said control information comprises ventilation control information having a ventilation mode and ventilation parameters of the ventilator.

37. The ventilator information display apparatus according to claim 36, wherein the target display information includes one or more of: patient information, monitoring information, control information, and layout information.

38. The ventilator information display apparatus according to claim 37, wherein the monitoring information includes one or more of: real-time values, short trends, long trends and mean values of physiological parameters of a patient.

39. The ventilator information display apparatus according to claim 37, wherein the control information includes one or more of: ventilation control information, equipment control information, and commonly used tool information.
